(19) United States
(12) Patent Application Publication
(10) Pub. No.: US 2019/0374146 A1
(43) Pub. Date: Dec. 12, 2019

(54) METHOD OF MAKING MICRONEEDLES, MOLD ASSEMBLY THEREFOR AND MICRONEEDLES ARRAY

(71) Applicant: SABIC GLOBAL TECHNOLOGIES B.V., Bergen op Zoom (NL)

(72) Inventors: Johannes DE BROUWER, Oisterwijk (NL); Herwig JUSTER, Bergen Op Zoom (NL)

(21) Appl. No.: 16/472,307

(22) PCT Filed: Dec. 21, 2017
(86) PCT No.: PCT/IB2017/058302

§ 371 Date: Jun. 21, 2019

Related U.S. Application Data
(60) Provisional application No. 62/437,553, filed on Dec. 21, 2016.

(51) Int. Cl. A61B 5/15; A61M 37/00; B29C 33/38; B29C 33/42

(52) U.S. Cl. CPC A61B 5/150282; A61M 37/0015; B29C 33/3842; B29C 33/42; B29L 2031/756; A61M 2207/10; A61M 2037/0023; A61M 2037/0046; A61M 2037/0053

(57) ABSTRACT

A mold assembly for forming a microneedle array exhibiting different aspect ratios is described. The mold assembly comprises a plurality of laminate mold layers 102, each of the laminate mold layers having a plurality of laminate recesses 104 and wherein the plurality of laminate recesses cooperate with an adjacent laminate mold layer to define a plurality of laminate cavities, and a base mold portion 111 having a base cavity configured to be disposed adjacent the plurality of laminate cavities such that the base cavity and the laminate cavities cooperate to define a mold for a microneedle array, wherein at least a portion of the plurality of laminate cavities vary in size relative to each other to provide a varying aspect ratio.

### TECHNICAL FIELD

The application concerns forming microneedle arrays wherein the array exhibits varying aspect ratios.

### BACKGROUND

Microneedles are attractive for delivery of certain therapeutics. These needles may be particularly desirable as a mode of therapeutic delivery because of the potential to replace syringe-with-needle type of injections with a pain free alternative. Microneedles can be virtually painless because they do not penetrate deep enough to contact nerves and only penetrate the outermost layer of the skin, unlike traditional syringes and hypodermic needles. Additionally, shallower penetration can also reduce the chance of infection or injury. Microneedles may also facilitate delivery of a more precise dosage of a therapeutic which enables the use of lower doses in treatments. Other advantages of microneedles for drug delivery include the simplified logistics (absence of required cold chain), ability for patient self-administration (no need for doctor, nurse, reduction of people transport). Beyond therapeutic delivery, drug delivery, microneedles have also been investigated for diagnostic applications. Bodily fluids coming out through the punctured skin can be analyzed for e.g. glucose or insulin.

Microneedles often require a manufacturing process that allows mass production at lowest cost, and as a consequence, shortest possible cycle time. In order to have proper transcription of mold texture and shape to the molded part, high flow may be necessary, especially having low viscosity at extremely high shear rates. Furthermore, good release from the production mold is important to reduce cycle time to improve the cost efficiency. These needles should have good strength to prevent breaking of the microneedle during usage. While there are a number of benefits to the use of microneedles and considerations with respect to forming them, certain challenges remain in microneedle production. It would be beneficial to prepare microneedles that exhibit an appropriate geometry for puncturing the skin.

### SUMMARY

Aspects of the present disclosure concern a mold assembly for forming a microneedle array, the mold assembly comprising: a laminate mold portion comprising a stack of laminate mold layers, each of the laminate mold layers having a plurality of laminate recesses and wherein the plurality of laminate recesses cooperate an adjacent laminate mold layer to define a plurality of laminate cavities; and a base mold portion having a base cavity configured to be disposed adjacent the plurality of laminate cavities such that the base cavity and the laminate cavities cooperate to define a mold for a microneedle array. At least a portion of the plurality of laminate cavities may vary in size relative to each other.

Other aspects concern methods of forming a microneedle array by contacting a laminate mold portion with a base mold portion, wherein the laminate mold portion comprises a plurality of laminate mold layers, each of the laminate mold layers comprising a plurality of laminate recesses, wherein the plurality of laminate recesses cooperate with an adjacent laminate mold layer to define a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

### BRIEF DESCRIPTION OF DRAWINGS

The above-mentioned and other features and advantages of this disclosure, and the manner of attaining them, will become apparent and be better understood by reference to the following description of one aspect of the disclosure in conjunction with the accompanying drawings, wherein:

FIG. 1A depicts a diagram of stacked laminate mold layers.

FIG. 1B depicts a diagram of interlocking stacked laminate mold layers according to aspects of the present disclosure.

FIG. 2 depicts a schematic diagram of varying geometries for a portion of a microneedle array prepared according to aspects of the present disclosure.

FIG. 3 depicts a three-dimensional diagram of a microneedle array prepared according to aspects of the present disclosure.

FIG. 4 presents the microneedle lengths obtained for six polymer materials used to form microneedle arrays according to aspects of the present disclosure.

FIG. 5 presents the microneedle lengths obtained for a second set of polymer materials used to form microneedle arrays according to aspects of the present disclosure.

FIG. 6 presents a schematic diagram representing laser microscopy and imaging results for microneedle array formed with a polymeric material according to aspects of the present disclosure.

FIG. 7 presents a schematic diagram representing angle measurements of the mold assembly for the microneedle array and the microneedle array formed therefrom.

FIG. 8 presents microneedle tip radius measurements for microneedles formed from PP PHC 31-81 material.

FIG. 9 presents microneedle tip radius measurements for microneedles formed from PC HPX8REU material.

FIG. 10 presents microneedle tip radius measurements for microneedles formed from Noryl™ HN731SE material.

FIG. 11 presents microneedle tip radius measurements for microneedles formed from ABS CYCOLAC™ HMG94MD material.

### DETAILED DESCRIPTION OF ILLUSTRATIVE EMBODIMENTS

The present disclosure can be understood more readily by reference to the following detailed description of the disclosure and the Examples included therein. Microneedles can be used to deliver a therapeutic or to draw interstitial fluids or blood without penetrating tissue as deep as traditional needles. Such microneedles can be used individually or as an array of needles. The needles are typically produced via mass production at a low cost. To efficiently function as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while the formed needles still maintain their structural integrity and strength. The mold assembly and methods of forming thereof may provide a microneedle array having the desired varying aspect ratio sufficient to provide a sharp tip among the microneedles and/or a sharp blade to properly penetrate or cut the skin. The mold assembly for forming a microneedle array may comprise a laminate mold portion and a base mold portion configured to form a microneedle array wherein at least a portion of the microneedles vary in size relative to each other.

According to aspects of the present disclosure, the mold assembly may comprise a laminate mold portion and a base mold portion. The laminate mold portion and the base mold portion may be positioned in a holder configured to contact the mold portions to form a mold for making a microneedle array.

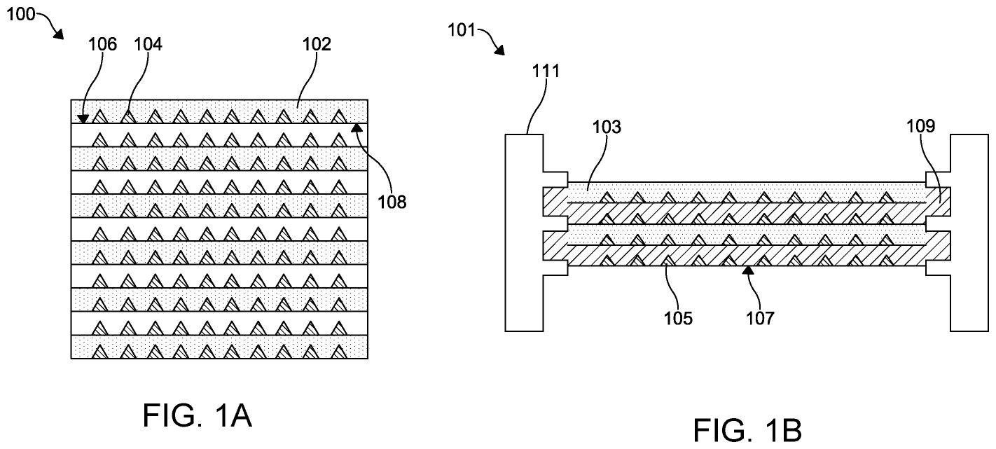

Referring to FIG. 1A, the laminate mold portion 100 may comprise a plurality of laminate mold layers 102, each layer comprising a plurality of laminate recesses 104 at a first surface 106 of each laminate mold layer. The plurality of mold layers 102 may be arranged in a stacked configuration such that each laminate recess 104 may be adjacent a second surface 108 of an adjacent laminate mold layer 102 to define a plurality of laminate cavities from the plurality of recesses 104 there between.

In certain aspects, the laminate mold layers may mechanically lock into the laminate mold holder to form the stacked configuration. One or more layers may have an interlocking feature to secure laminate mold layers to the laminate mold holder thereby forming the stacked configuration. Referring to FIG. 1B, the laminate mold portion 101 may comprise a plurality of laminate mold layers 103, each laminate mold layer 103 comprising a plurality of laminate recesses 105 at a first surface 107 of each laminate mold layer 103. In an example, alternating layers of the laminate mold layers 103 may comprise an interlocking feature, such as a shoulder 109, to secure the laminate mold layer into a mold holder 111. Mechanical locking of the laminate mold layers leads to a precise replication and enables passive venting of trapped gases within the mold assembly to provide the desired geometries.

The laminate mold layers may be manufactured by an appropriately precise process to facilitate interlocking of the layers. The laminate mold layers may be manufactured precisely according to a low voltage electrical discharge machining (EDM) process. The laminate mold layers may be formed from a material that is resilient to the EDM process. As an example the laminate mold layer may be formed from stainless steel, such as, for example, Grade 420 stainless steel. In various aspects, the laminate mold portion may comprise up to about 21 laminate mold layers. As a specific example, the laminate mold portion may comprise eleven laminate mold layers.

The laminate mold portion may comprise a plurality of laminate mold layers. Each of the laminate mold layers may include a plurality of laminate recesses. The laminate recesses may be disposed throughout the laminate mold layers such that the plurality of laminate recesses contact, or cooperate with, an adjacent laminate mold layer to define a plurality of laminate cavities. The stacked configuration of the plurality of laminate mold layers may allow the plurality of recesses of each laminate mold layer to form a plurality of cavities. In some aspects, each laminate mold layer may comprise up to about 50 laminate recesses, which may form up to about 50 laminate cavities in the stacked configuration of the mold layers. In a specific example, each laminate mold layer may comprise 10 laminate recesses.

As described each laminate recess may be adjacent a surface of an adjacent laminate mold layer, thereby forming a laminate cavity. Each laminate cavity may have a particular geometry which corresponds to the shape of a microneedle in the microneedle array. At least a portion of the laminate cavities exhibit a half-pyramid geometry where two side lengths of the half-pyramid form an apex, corresponding to a penetrative point of a microneedle formed in the mold. Each laminate cavity may thus have a certain base size and apex angle.

At least a portion of the plurality of laminate cavities may varies in size relative to each other. This variation in size creates a varying aspect ratio in the microneedle array. For example, side lengths of the half-pyramid geometry of each laminate cavity may vary. At least a first portion of the laminate cavities may have a side length of up to about 0.8 millimeters (mm) while at least a second portion of the laminate cavities may have side length of up to about 1.0 mm. The varying side lengths of the laminate cavities may ensure that the base size of the laminate cavities also varies. For laminate cavities having a side length of 0.8 mm or about 0.8 mm, the base size of the laminate cavity may vary from 0.3 mm to 15 mm or from about 0.3 mm to about 0.15 mm, specifically from 0.0342 mm to 0.174 mm or from about 0.342 mm to about 0.174 mm. For laminate cavities having a side length of about 1.0 mm, the base may vary from 0.25 mm to 0.15 mm or from about 0.25 mm to about 0.15 mm, specifically from 0.0173 mm to 0.273 mm or from about 0.173 mm to about 0.273 mm. The base size may vary according to the apex angle size for the half-pyramid geometry of the side length may be up to about 1.0 mm. In certain aspects, the apex angle size may vary from 20° C. to 10° or from about 20° to about 10°.

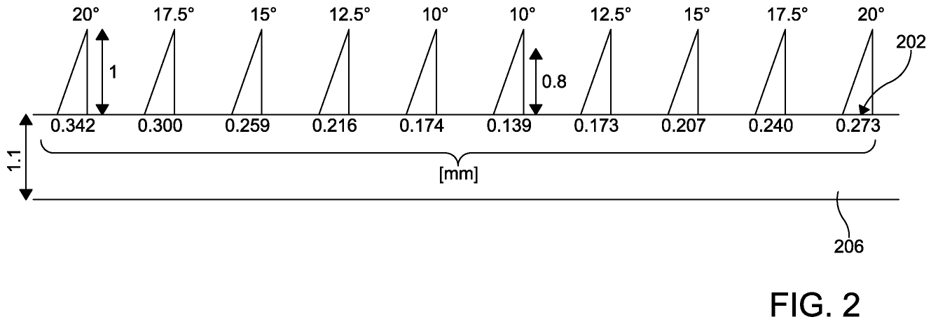

Generally, the smaller the apex angle, the smaller the base size. The varying side length, base size, and apex angle of the half-pyramid laminate cavities may result in a varying aspect ratio among the microneedle array. FIG. 2 presents a diagram of cross section of a microneedle array formed according to the methods described herein and exhibiting varying aspect ratios according to the changing base size 202 and apex angle 204 as described above. Over the variation of the bases, different aspect ratios from about 1:2 to about 1:4 may be apparent within the microneedle array. The varying aspect ratios may allow for different cutting or penetrative profiles of microneedles formed using the mold assembly described herein.

As provided, the laminate mold portion comprising the laminate mold layers and the laminate cavities thereof may be contacted with the base mold portion to define a mold for forming a microneedle array. The base mold portion may comprise a base cavity. The base cavity of the base mold portion may be configured to be disposed adjacent the laminate cavities of the laminate mold layers when the base mold portion and the laminate mold portion are brought into contact to define a mold for the microneedle array. That is, the defined mold for the microneedle array comprises an interior formed by the base cavity and the plurality of laminate cavities. The base cavity may be so configured to form a base for the microneedle array. The base cavity may have a depth of from 0.8 mm to 1.1 mm or about 0.8 mm to about 1.1 mm. See also FIG. 2, wherein the depth of the base cavity 206 is 1.1 mm or about 1.1 mm.

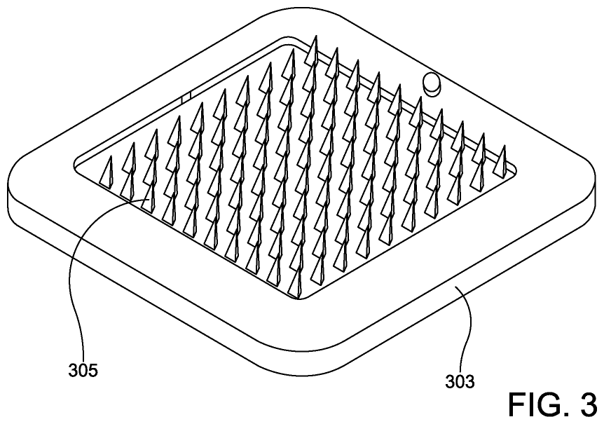

In various aspects of the present disclosure, a microneedle array may be formed as shown in FIG. 3 depicting a three-dimensional view of the microneedle array comprising a base 303 and a plurality of microneedles 305. To further demonstrate varying aspect ratios which may be achieved according to the disclosed methods, a series of five rows of ten microneedles on a lower left portion of the array may have a half-pyramid side length of 1 mm while a series of five rows often microneedles on upper or right portion of the array have a half-pyramid side length of 0.8 mm or about 0.8 mm.

In a specific example, each ejector pin may have a diameter of 1.5 mm (or about 1.5 mm) and pushes the array out after the cooling process has finished and the mold is opened again. Furthermore, submicron venting between the laminates helps to replicate microstructures due to better filling behavior. The venting has a passive working principle.

The mold portions (laminate mold portion and base mold portion) may be comprised of any material sufficient to withstand heating and to receive a heated replication material desired to form the microneedle array. In one example, the mold portions may be comprised of stainless steel having a surface roughness of 0.3 micrometers (μm) or about 0.3 μm.

The mechanical locking of the stacked laminates may lead to a precise replication and still allows passive venting of the desired geometries. The key is the precise manufacturing of the laminates using a low voltage EDM process. Therefore, the presented insert method shows a fast and cost saving approach in the product development of engineering thermoplastic based microneedle arrays. Moreover, the stacked laminate configuration described herein may facilitate the expulsion of any trapped gas within the mold assembly.

A microneedle array as described herein may be formed by a method comprising contacting a laminate mold portion with a base mold portion of a mold assembly. The laminate mold portion may comprise a plurality of laminate mold layers, each of the laminate mold layers comprising a plurality of laminate cavities. The base mold portion may comprise a base cavity configured to be disposed adjacent the plurality of laminate cavities such that the base cavity and the laminate cavities cooperate to define a mold cavity for a microneedle array. A material may then be contacted with the mold cavity to form an array of microneedles comprising the material, wherein at least a portion of the array of microneedles exhibit varying aspect ratios. The molding process to form the microneedle array may be achieved by inserting the mold assembly comprising the laminate mold portion and base mold portion into any convention polymer molding apparatus.

In certain aspects, the mold assembly may be disposed within a mold holder such that the laminate mold portion may be stationary while the base mold portion is movable within the holder to allow contacting of the two mold portions. The mold holder may be configured to bring the base portion and the laminate mold portion into contact. Moreover, the mold holder may provide uptake and guiding of ejection pins configured around the mold assembly to form the microneedle array.

As provided, microneedles of the microneedle array of the present disclosure may be used to deliver a therapeutic or to draw interstitial fluids or blood without penetrating tissue as deep a traditional needles. The microneedles may be used individually or as an array of needles. The size of such needles typically is measured in microns. Some microneedles are between 100 μm and 1 mm in length, preferably between 10 μm and 500 μm, more preferably between 30 μm and 200 μm and more preferably between 100 μm and 150 μm. The needles are typically produced via mass production at a low cost. To efficiently function as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while the formed needles still maintain their structural integrity and strength. The mold assembly and methods of forming thereof may provide a microneedle array having the desired varying aspect ratio sufficient to provide a sharp tip among the microneedles and a sharp blade to properly penetrate or cut the skin. A mold assembly for forming a microneedle array may comprise a laminate mold portion and a base mold portion. The laminate mold portion may comprise a plurality of laminate mold layers, each of the laminate mold layers having a plurality of laminate recesses. The plurality of laminate recesses may cooperate with an adjacent laminate mold layer to define a plurality of laminate cavities. The base mold portion may have a base cavity configured to be disposed adjacent the plurality of laminate cavities such that the base cavity and the laminate cavities cooperate to define a mold for a microneedle array. To provide a microneedle array having a varying aspect ratio, at least a portion of the plurality of laminate cavities may vary in size relative to each other.

A microneedle array as formed in the present disclosure may comprise solid microneedles. In an aspect, for therapeutic delivery via a solid microneedle array, the therapeutic may be coated onto the microneedles and dissolves or diffuses. That is, active components of the therapeutic may dissolve or diffuse when the microneedles penetrate skin, allowing interstitial fluid to contact the drug formulation. In this way, the therapeutic may be released just below the skin. Microneedles formed herein should have sufficient mechanical strength to remain intact (i) while being inserted into the biological barrier, (ii) while remaining in place for up to a number of days, and (iii) while being removed.

Microneedles may be manufactured via commercial molding technology. A microneedle array may be formed using the mold assembly of the present disclosure. In one aspect, the mold assembly comprising the laminate mold portion and base mold portion may be inserted in a conventional injection molding apparatus for forming a microneedle array. As an example, the mold assembly may be inserted into an injection molding apparatus. In a single injection molding cycle, a microneedle array having differing aspect ratios may be formed. The differing aspect ratios of the mold assembly may allow a user to evaluate a preferred replication material for forming the microneedle array therein. A user may be able to estimate the best replication material for use in the mold assembly for the microneedle array based on filling and overall processing behavior of a substrate or material.

In various aspects, the substrate may comprise a polymer material. The substrate for forming a microneedle array using the disclosed mold assembly may comprise a polymer or a mixture of polymers. Generally, the polymer mixture may be supplied in a liquid or flowable state, via for example, an extrusion die apparatus, to the mold assembly. A solid product comprising the microneedle array may then be obtained from the mold assembly after cooling. Exemplary polymer materials may comprise engineering thermoplastics such as polycarbonates, polyetherimides, polyphenylene ether, liquid crystalline polymers and polybutylene terephthalate, as well as blends of polycarbonate with acrylic butadiene styrene plastics.

The polymer material for forming the microneedle array may further comprise one or more additives intended to impart certain characteristics to a microneedle array formed by the mold assembly described herein. The polymer material may include one or more of an impact modifier, flow modifier, antioxidant, heat stabilizer, light stabilizer, ultraviolet (UV) light stabilizer, UV absorbing additive, plasticizer, lubricant, antistatic agent, antimicrobial agent, colorant (e.g., a dye or pigment), surface effect additive, radiation stabilizer, or a combination comprising one or more of the foregoing. For example, a combination of a heat stabilizer, and ultraviolet light stabilizer can be used. In general, the additives are used in the amounts generally known to be effective. For example, the total amount of the additive composition can be 0.001 wt % to 10.0 wt %, or about 0.001 wt % to about 10 wt %, or 0.01 wt % to 5 wt %, or about 0.01 wt % to about 5 wt %, each based on the total weight of all ingredients in the composition.

The polymer material may include various additives ordinarily incorporated into polymer compositions, with the proviso that the additive(s) are selected so as to not significantly adversely affect the desired properties of the thermoplastic composition (good compatibility for example). Such additives can be mixed at a suitable time during the mixing of the components for forming the composition.

In addition, the polymer material may exhibit excellent release, as measured by ejection force (N) and coefficient of friction. The polymer material also preferably show (i) high flow at high shear conditions to allow good transcription of mold texture and excellent filling of the finest mold features, (ii) good strength and impact (as indicated by ductile Izod Notched Impact at room temperature and modulus), and (iii) high release to have efficient de-molding and reduced cooling and cycle time during molding. The microneedles formed herein may have sufficient mechanical strength to remain intact (i) while being inserted into the biological barrier, (ii) while remaining in place for up to a number of days, and (iii) while being removed

### Definitions

It is to be understood that the terminology used herein is for the purpose of describing particular aspects only and is not intended to be limiting. As used in the specification and in the claims, the term “comprising” can include the embodiments “consisting of” and “consisting essentially of.” Unless defined otherwise, all technical and scientific terms used herein have the same meaning as commonly understood by one of ordinary skill in the art to which this disclosure belongs. In this specification and in the claims that follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “weight %,” and “wt. %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {M}_{i}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si—O—Si linkage.

The term “flowable” means capable of flowing or being flowed. Typically a polymer is heated such that it is in a melted state to become flowable. ° C. is degrees Celsius. μm is micrometer.

Aspects

The present disclosure comprises at least the following aspects.

Aspect 1A. A mold assembly for forming a microneedle array, the mold assembly comprising: a laminate mold portion comprising a plurality of laminate mold layers, each of the laminate mold layers having a plurality of laminate recesses and wherein the plurality of laminate recesses cooperate an adjacent laminate mold layer to define a plurality of laminate cavities; and a base mold portion having a base cavity configured to be disposed adjacent the plurality of laminate cavities such that the base cavity and the laminate cavities cooperate to define a mold for a microneedle array; and wherein at least a portion of the plurality of laminate cavities vary in size relative to each other to provide a varying aspect ratio.

Aspect 1B. A mold assembly for forming a microneedle array, the mold assembly consisting essentially of: a laminate mold portion comprising a plurality of laminate mold layers, each of the laminate mold layers having a plurality of laminate recesses and wherein the plurality of laminate recesses cooperate an adjacent laminate mold layer to define a plurality of laminate cavities; and a base mold portion having a base cavity configured to be disposed adjacent the plurality of laminate cavities such that the base cavity and the laminate cavities cooperate to define a mold for a microneedle array; and wherein at least a portion of the plurality of laminate cavities vary in size relative to each other to provide a varying aspect ratio.

Aspect 1C. A mold assembly for forming a microneedle array, the mold assembly consisting of: a laminate mold portion comprising a plurality of laminate mold layers, each of the laminate mold layers having a plurality of laminate recesses and wherein the plurality of laminate recesses cooperate an adjacent laminate mold layer to define a plurality of laminate cavities; and a base mold portion having a base cavity configured to be disposed adjacent the plurality of laminate cavities such that the base cavity and the laminate cavities cooperate to define a mold for a microneedle array; and wherein at least a portion of the plurality of laminate cavities vary in size relative to each other to provide a varying aspect ratio.

Aspect 2. The mold assembly of any of aspects 1A-1C, wherein one or more layers of the plurality of laminate mold layers include an interlocking feature to secure the plurality of mold layers forming to laminate mold portion into a mold holder.

Aspect 3. The mold assembly of aspect 2, wherein the laminate mold portion and the base mold portion are disposed within a mold holder.

Aspect 4. The mold assembly of any one of aspects 1A-3, wherein at least a portion of the plurality of the laminate cavities has a half-pyramid geometry, wherein two side lengths of the half-pyramid geometry form an apex of each half-pyramid, and wherein at least a portion of the laminate cavities exhibit varying side lengths.

Aspect 5. The mold assembly of any one of aspects 1A-4, wherein the laminate mold portion comprises up to about twenty-one laminate mold layers.

Aspect 6. The mold assembly of any one of aspects 1A-5, wherein each laminate mold layer comprises up to about 50 laminate cavities.

Aspect 6. The mold assembly of any one of aspects 1A-5, wherein each laminate mold layer comprises up to 50 laminate cavities.

Aspect 7. The mold assembly of any one of aspects 1A-6, wherein at least a portion of the plurality of laminate cavities has two side lengths of up to about 1.0 mm.

Aspect 7. The mold assembly of any one of aspects 1A-6, wherein at least a portion of the plurality of laminate cavities has two side lengths of up to 1.0 mm.

Aspect 8. The mold assembly of any one of aspects 1A-7, wherein at least a portion of the plurality of cavities have two side lengths of up to 0.8 mm.

Aspect 8. The mold assembly of any one of aspects 1A-7, wherein at least a portion of the plurality of cavities have two side lengths of up to about 0.8 mm.

Aspect 9. The mold assembly of any one of aspects 1A-8, wherein the plurality of laminate cavities exhibit aspect ratios varying between 1:2 and 1:4.

Aspect 10. The mold assembly of any one of aspects 1A-9, wherein laminate mold layers comprise steel.

Aspect 11. The mold assembly of any one of aspects 1A-10, wherein the plurality of laminate mold layers facilitate expulsion of trapped gas in the mold assembly.

Aspect 12. The mold assembly any one of aspects 1A-11, wherein the mold assembly is disposed within an injection molding assembly.

Aspect 13. The mold assembly of any one of aspects 1A-12, wherein the plurality of laminate mold layers comprises eleven laminate mold layers.

Aspect 14. The mold assembly of any one of aspects 1A-13, wherein the plurality of laminate cavities comprises ten cavities.

Aspect 15. The mold assembly of any one of aspects 1A-14 wherein the laminate mold portion is disposed within a first portion of a mold holder and the base mold portion is disposed within a second portion of a mold holder and wherein the second holder is configured to contact the first holder thereby contacting the laminate mold portion with the base mold portion.

Aspect 16A. A method of forming a microneedle array comprising: contacting a laminate mold portion with a base mold portion, wherein the laminate mold portion comprises a plurality of laminate mold layers, each of the laminate mold layers comprising a plurality of laminate recesses, wherein the plurality of laminate recesses cooperate with an adjacent laminate mold layer to define a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

Aspect 16B. A method of forming a microneedle array consisting essentially of: contacting a laminate mold portion with a base mold portion, wherein the laminate mold portion comprises a plurality of laminate mold layers, each of the laminate mold layers comprising a plurality of laminate recesses, wherein the plurality of laminate recesses cooperate with an adjacent laminate mold layer to define a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

Aspect 16C. A method of forming a microneedle array consisting of: contacting a laminate mold portion with a base mold portion, wherein the laminate mold portion comprises a plurality of laminate mold layers, each of the laminate mold layers comprising a plurality of laminate recesses, wherein the plurality of laminate recesses cooperate with an adjacent laminate mold layer to define a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

Aspect 17. The method of any of aspects 16A-16C, wherein one or more layers of the plurality of laminate mold layers includes an interlocking feature to secure the plurality of mold layers that form the laminate mold portion into a mold holder.

Aspect 18. The method of any one of aspect 16A-17, wherein the plurality of laminate mold layers facilitates expulsion of trapped gas in the mold assembly via interlocking among the plurality of laminate mold layers.

Aspect 19. The method of any one of aspects 16A-18, wherein at least a portion of the plurality of laminate cavities exhibit a half-pyramid geometry.

Aspect 20. The method of aspect 19, wherein the plurality of laminate cavities has varying geometry for the half-pyramid geometries.

Aspect 21. The method of any one of aspects 16A-20, wherein the plurality of laminate mold layers comprises eleven laminate mold layers.

Aspect 22. The method of any one of aspects 16A-21, wherein the polymeric material is heated to a temperature above a melting temperature of the polymeric material prior to contacting with the mold cavity.

Aspect 23. The method of any one of aspects 16A-22, wherein the laminate mold layers are formed by a process of electro discharge machining.

Aspect 24. The method of any one of aspects 16A-23, wherein the plurality of laminate mold layers facilitates expulsion of trapped gas in the mold assembly.

Aspect 25. The method of any one of aspects 16A-24, wherein the contacting of the polymeric material with the mold cavity comprises an injection molding process.

Aspect 26. The method of any one of aspects 16A-25, wherein the polymeric material comprises polycarbonate, polyetherimide, polyphenylene ether, polybutylene terephthalate, or combinations thereof.

Aspect 27A. A microneedle array formed by a method comprising: contacting a laminate mold portion with a base mold portion, wherein the laminate mold portion comprises a plurality of laminate mold layers, each of the laminate mold layers comprising a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

Aspect 27B. A microneedle array formed by a method consisting essentially of: contacting a laminate mold portion with a base mold portion, wherein the laminate mold portion comprises a plurality of laminate mold layers, each of the laminate mold layers comprising a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

Aspect 27. A microneedle array formed by a method consisting of: contacting a laminate mold portion with a base mold portion, wherein the laminate mold portion comprises a plurality of laminate mold layers, each of the laminate mold layers comprising a plurality of laminate cavities, wherein the base mold position comprises a base cavity configured to be disposed adjacent the plurality of laminate cavities such that the base cavity and the laminate cavities cooperate to define a mold cavity for a microneedle array; and contacting a polymeric material with the mold cavity to form an array of microneedles comprising the polymeric material, wherein at least a portion of the array of microneedles exhibit varying aspect ratios.

### Examples

The disclosure is illustrated by the following non-limiting examples. For evaluating the laminate structure and the capability of replicating the microneedle structure, injection molding trials were performed at the Centre for Polymer Micro and Nano Technology, University Bradford, UK. The laminate mold portions were produced by ISOMETRIC™ Inc.

For testing the functionality of the inserts, six different materials were chosen (PHC 31-81, LEXAN™ HPX8REU, VALOX™ HX312C, CYCOLOY™ HCX 1640, NORYL™HN731SE, ULTEM™ HU1010), CYCOLAC HMG94MD, and Ticona Vectra B230. The materials were used to form microneedle arrays. Table 1 presents the details for the materials.

TABLE 1
Materials for molding.

| | |
|---|---|
| PHC 31-81 | Poly (propylene-ethylene) [CASRN 9010-79-1] |
| LEXAN ™ HPX8REU | blend of Polycarbonate [CASRN 111211-39-3]/ |
| | Polycarbonate-Siloxane copolymer |
| | [CASRN 202483-49-6] |
| VALOX ™ HX312C | Poly (butylene terephthalate) |
| | [CASRN 30965-26-5] |
| CYCOLOY ™ | Polycarbonate [CASRN 25929-04-8]/ |
| HCX1640 | ABS [CASRN 9003-56-9] blend |
| NORYL ™HN731SE | Polyphenylene ether [CASRN 25134-01-4]/ |
| | High impact polystyrene [CASRN 9003-55-8] |
| | and/or polystyrene [CASRN 9003-53-6] blend |
| ULTEM ™ HU1010 | Polyetherimide [CASRN 61128-46-9] |
| CYCOLAC ™ | Acrylonitrile butadiene styrene (ABS) resin |
| HMG94MD | |
| Ticona VECTRA ™ | 1,4-benzenediacarboxlyic acid, polymer with |
| B230 | 6(acetyloxy)-2-naphtalene carboxylic acid and |
| | {4(acetoxy)phenyl} acetamide |
| | [CASRN 82538-13-4] |

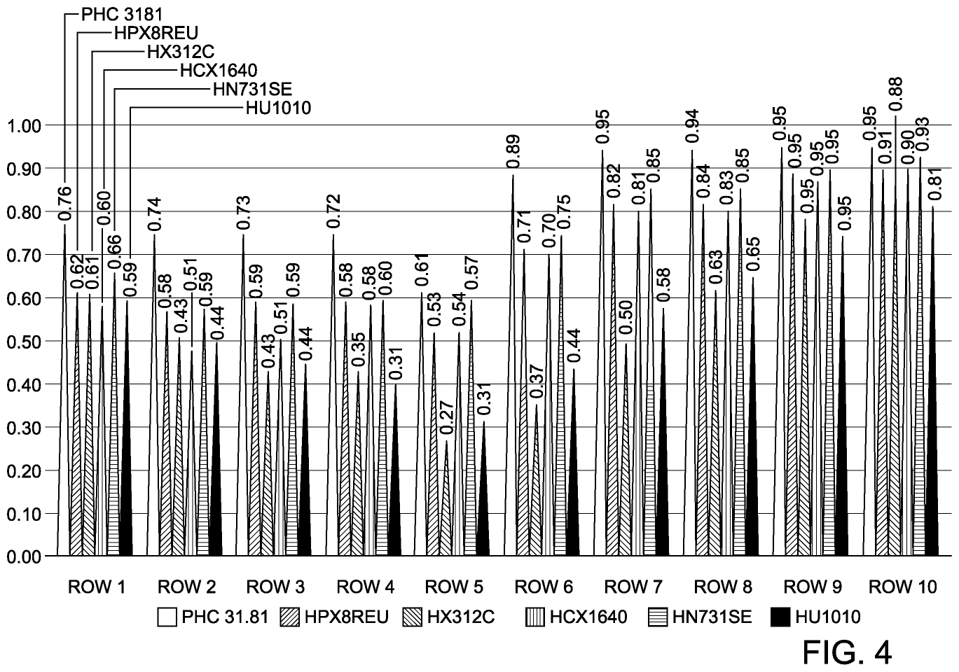

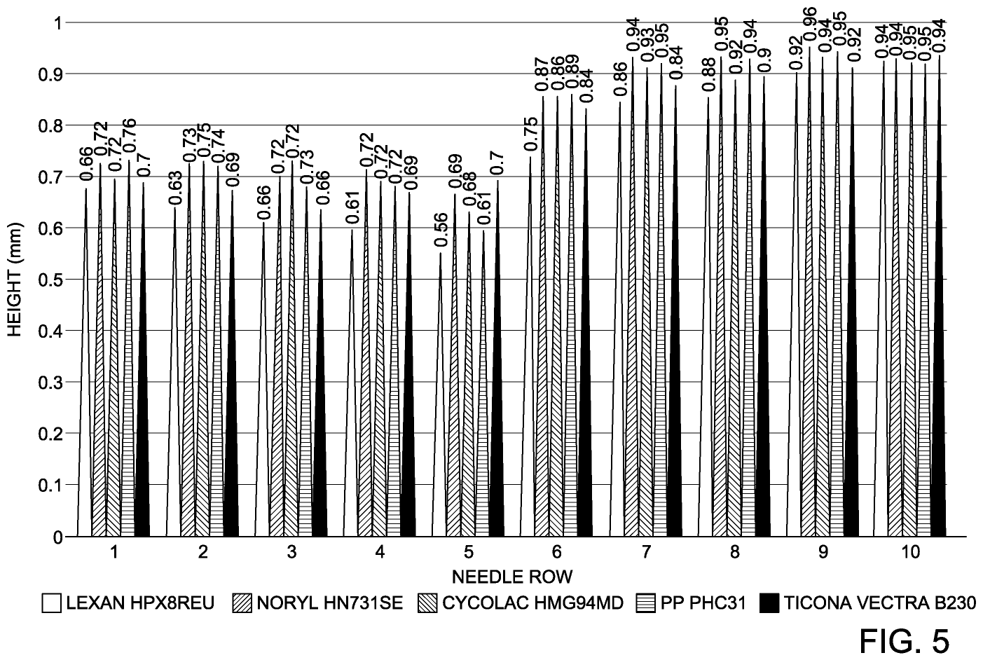

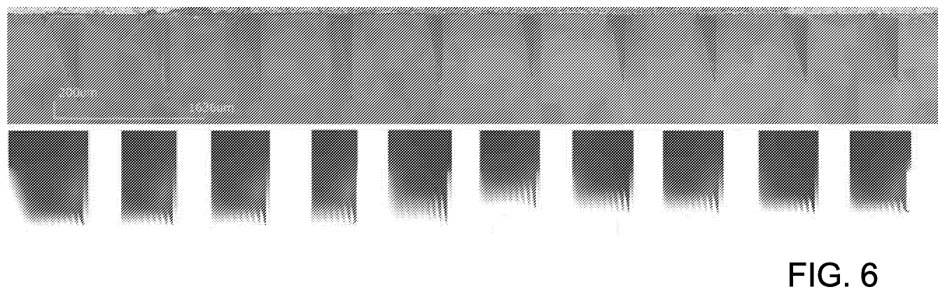

The materials were processed using an injection molding machine (Wittman Battenfeld MicroPower 15). Evaluation of the length of formed microneedles was measured using laser microscopy. As an example, the results for microneedle lengths are presented in FIG. 4. Rows 1 through 5 show varying microneedle lengths between about 0.25 mm and about 0.8 mm corresponding to five rows of the microneedle array. The second set of rows, Rows 6 through 10 show varying microneedle lengths between about 0.3 and about 1.0 mm. An additional array of lengths are shown in FIG. 5 for another set of materials forming the microneedles. Rows 1 through 5 show varying microneedle lengths between about 0.5 mm and about 0.8 mm corresponding to five rows of the microneedle array. The second set of rows, Rows 6 through 10 show varying microneedle lengths between about 0.7 and about 1.0 mm. Analysis diagrams of microneedle measurements (200 μm and 1635 μm) are presented in FIG. 6 for PP PHC 31-81.

Table 2 shows the numerical values of the laser microscopy and color camera measurements PP HPC 31-81 showed good filling of all microneedle cavities, but the tips of the needles were easily damaged or bent.

TABLE 1
Measured length of the PP PHC 31-81 using
laser microscopy and color camera system.

| Confocal | Color camera | Difference | Difference to |
|---|---|---|---|
| (mm) | (mm) | (mm) | desired |
| 0.736 | 0.740 | −0.004 | 0.064 |
| 0.759 | 0.760 | −0.001 | 0.041 |
| 0.755 | 0.761 | −0.006 | 0.045 |
| 0.749 | 0.758 | −0.009 | 0.051 |
| 0.672 | 0.685 | −0.012 | 0.128 |
| 0.906 | 0.920 | −0.013 | 0.094 |
| 0.933 | 0.943 | −0.009 | 0.094 |
| 0.945 | 0.955 | −0.010 | 0.055 |
| 0.947 | 0.950 | −0.003 | 0.053 |
| 0.935 | 0.941 | −0.005 | 0.065 |

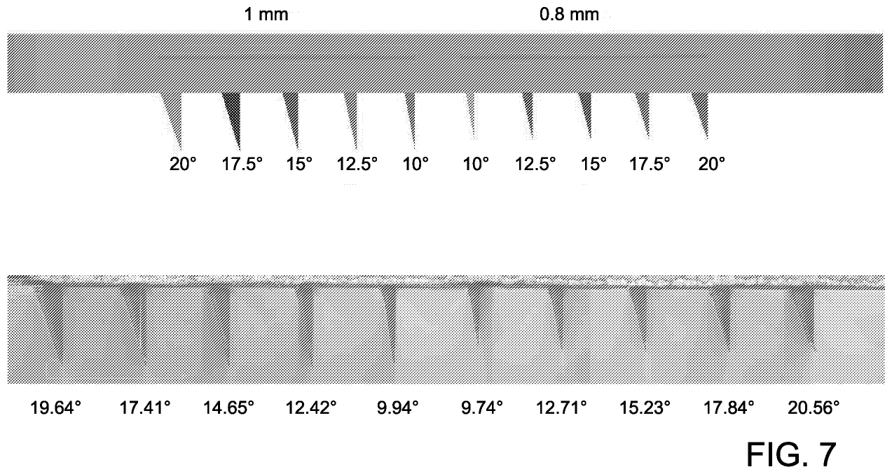

Angle measurements were also performed for formed microneedles. Apart of filling the array, correct replication of each needle on the array was investigated. For this, an angle measurement of the needle was done. The angle is formed by the vertical 90° straight side together with the inclined side forming the tip. Results for PP PHC 31-81 are shown in FIG. 7. The results were compared with the CAD model and accordance could be found. The formed microneedle array appeared consistent with the measured angles of the mold assembly for the microneedle array.

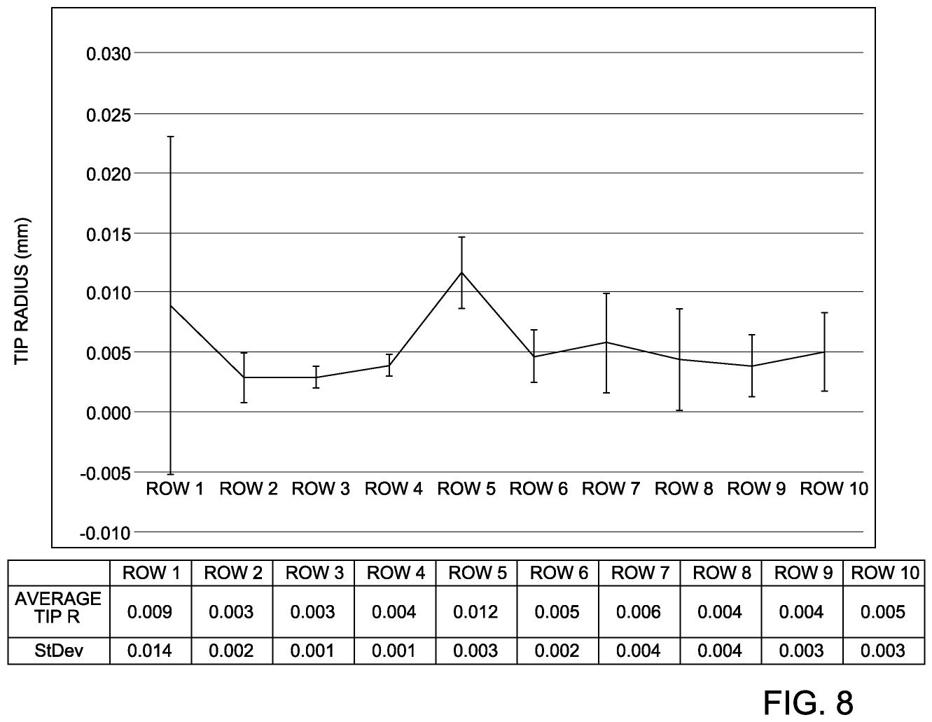

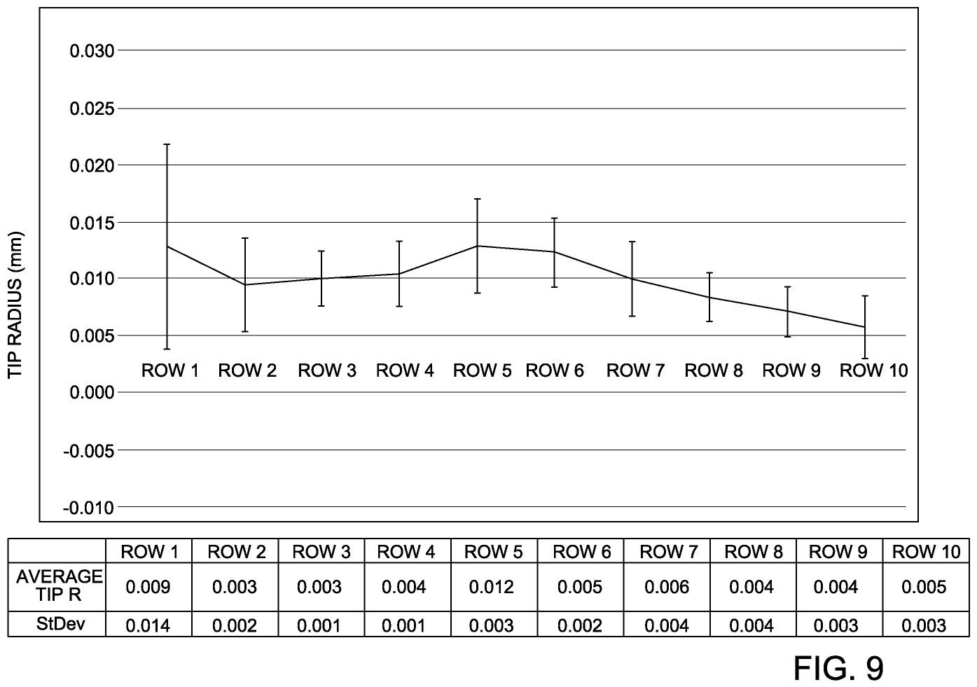

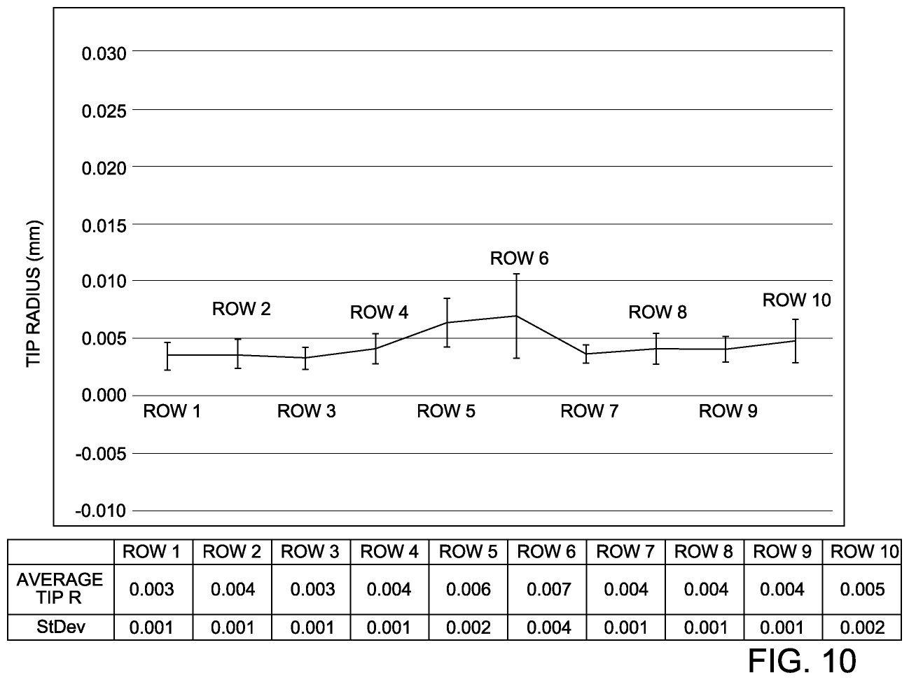

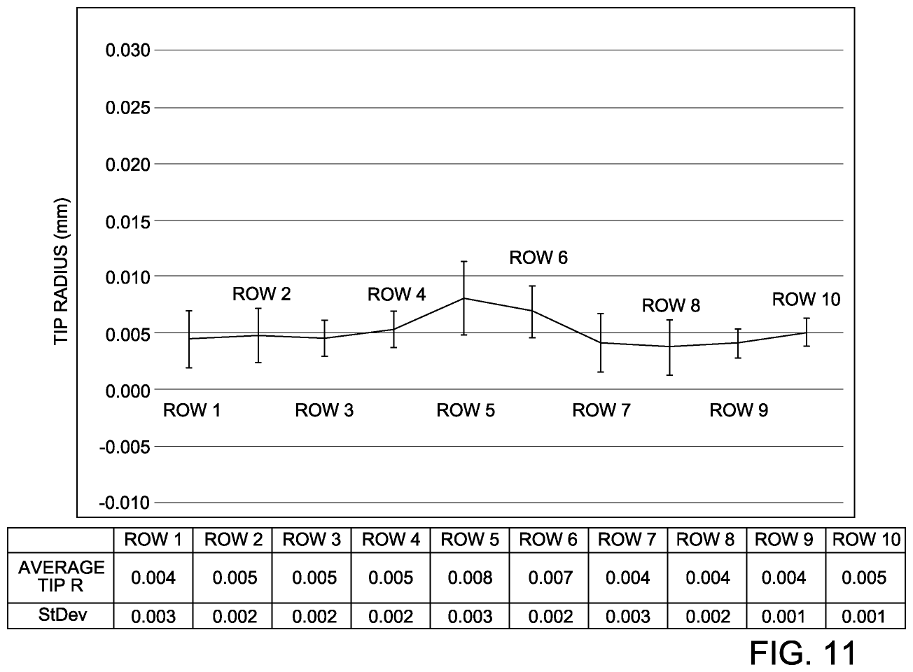

The tip radius for microneedles was also measured for varying samples (arranged according to the row of the microneedle array, for a total of 10 rows of microneedles). The tip radius measurements are provided for PP PHC 31-81, PC HPX8REU, Noryl HN731SE, and CYCOLAC HMG94MD in FIGS. 8-11, respectively. The tips appeared to be somewhat consistent given the standard deviation.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A mold assembly for forming a microneedle array, the mold assembly comprising:
a laminate mold portion comprising a plurality of laminate mold layers, each of the laminate mold layers having a plurality of laminate recesses and wherein the plurality of laminate recesses cooperate with an adjacent laminate mold layer to define a plurality of laminate cavities; and
a base mold portion having a base cavity configured to be disposed adjacent the plurality of laminate cavities such that the base cavity and the laminate cavities cooperate to define a mold for a microneedle array,
wherein at least a portion of the plurality of laminate cavities vary in size relative to each other to provide a varying aspect ratio, and
wherein at least a portion of the plurality of the laminate cavities has a half-pyramid geometry, wherein two side lengths of the half-pyramid geometry form an apex of each half-pyramid, and wherein at least a portion of the laminate cavities exhibit varying side lengths.

2. The mold assembly of claim 1, wherein one or more layers of the plurality of laminate mold layers includes an interlocking feature to secure the plurality of laminate mold layers that form the laminate mold portion into a mold holder.

3. The mold assembly of claim 2, wherein the laminate mold portion and the base mold portion are disposed within a mold holder.

4. (canceled)

5. The mold assembly of claim 1, wherein the laminate mold portion comprises up to about twenty-one laminate mold layers.

6. The mold assembly of claim 1, wherein each laminate mold layer comprises up to about 50 laminate cavities.

7. The mold assembly of claim 1, wherein at least a portion of the plurality of laminate cavities has two side lengths of up to about 1.0 mm.

8. The mold assembly of claim 1, wherein the plurality of laminate cavities exhibit aspect ratios varying between 1:2 and 1:4.

9. The mold assembly of claim 1, wherein the plurality of laminate mold layers facilitate expulsion of trapped gas in the mold assembly.

10. The mold assembly claim 1, wherein the mold assembly is disposed within an injection molding assembly.

11. A method of forming a microneedle array comprising:
contacting a laminate mold portion with a base mold portion to form a mold assembly,
wherein the laminate mold portion comprises a plurality of laminate mold layers, each of the laminate mold layers comprising a plurality of laminate recesses, wherein the plurality of laminate recesses cooperate with an adjacent laminate mold layer to define a plurality of laminate cavities,
wherein the base mold portion comprises a base cavity configured to be disposed adjacent the plurality of laminate cavities such that the base cavity and the laminate cavities cooperate to define a mold cavity for a microneedle array; and
contacting a polymeric material with the mold cavity to form an array of microneedles comprising the polymeric material, wherein at least a portion of the array of microneedles exhibit varying aspect ratios,
wherein at least a portion of the plurality of laminate cavities exhibit a half-pyramid geometry and wherein the plurality of laminate cavities has varying geometry for the half-pyramid geometries.

12. The method of claim 11, wherein one or more layers of the plurality of laminate mold layers includes an interlocking feature to secure the plurality of laminate mold layers that form the laminate mold portion into a mold holder.

13. The method of claim 11, wherein the plurality of laminate mold layers facilitates expulsion of trapped gas in the mold assembly via interlocking among the plurality of laminate mold layers.

14. (canceled)

15. (canceled)

16. The method of claim 11, wherein the plurality of laminate mold layers comprises eleven laminate mold layers.

17. The method of claim 11, wherein the polymeric material is heated to a temperature above a melting temperature of the polymeric material prior to contacting with the mold cavity.

18. The method of claim 11, wherein the laminate mold layers are formed by a process of electro discharge machining.

19. The method of claim 11, wherein the polymeric material comprises polycarbonate, polyetherimide, polyphenylene ether, polybutylene terephthalate, or combinations thereof.

20. A microneedle array formed by a method comprising:
contacting a laminate mold portion with a base mold portion,
wherein the laminate mold portion comprises a plurality of laminate mold layers, each of the laminate mold layers comprising a plurality of laminate cavities,
wherein the base mold portion comprises a base cavity configured to be disposed adjacent the plurality of laminate cavities such that the base cavity and the laminate cavities define a mold cavity for a microneedle array; and
contacting a polymeric material with the mold cavity to form an array of microneedles comprising the polymeric material, wherein at least a portion of the array of microneedles exhibit varying aspect ratios,
wherein at least a portion of the plurality of the laminate cavities has a half-pyramid geometry, wherein two side lengths of the half-pyramid geometry form an apex of each half-pyramid, and wherein at least a portion of the laminate cavities exhibit varying side lengths.
